(19) United States
(12) Patent Application Publication
(10) Pub. No.: US 2026/0307066 A1
(43) Pub. Date: Oct. 8, 2026

(54) BIFUNCTIONAL ACIDIC CATALYSTS AND PREPERATION METHODS OF FURFURAL VIA CATALYZING HEMICELLULOSE OR XYLOSE

(71) Applicants: ZHEJIANG HUAKANG PHARMACEUTICAL CO., LTD., Quzhou (CN); NANJING UNIVERSITY, Nanjing (CN)

(72) Inventors: Youting WU, Nanjing (CN); Mian LI, Quzhou (CN); Guangzhi XU, Nanjing (CN); Chengjun LIAO, Quzhou (CN); Changhui HU, Quzhou (CN); Xingbang HU, Nanjing (CN); Xinping CHENG, Quzhou (CN); Zhuoheng TU, Nanjing (CN); Deshui CHEN, Quzhou (CN)

(73) Assignees: ZHEJIANG HUAKANG PHARMACEUTICAL CO., LTD., Quzhou (CN); NANJING UNIVERSITY, Nanjing (CN)

(21) Appl. No.: 19/457,573

(22) Filed: Jan. 23, 2026

Related U.S. Application Data
(63) Continuation of application No. PCT/CN2024/095618, filed on May 27, 2024.

(30) Foreign Application Priority Data
Jul. 23, 2023 (CN) 202310907836.9

(51) Int. Cl. B01J 31/02; B01J 27/10; B01J 31/04; B01J 37/04; C07D 307/50

(52) U.S. Cl. CPC B01J 31/0239; B01J 27/10; B01J 31/04; B01J 37/04; C07D 307/50; B01J 2231/76

(57) ABSTRACT

A bifunctional acidic catalyst and a preparation method of furfural via catalyzing hemicellulose or xylose are provided. The bifunctional acidic catalyst includes choline chloride, Brønsted acid, and Lewis acid in a molar ratio of 1:1:(1-4), and the bifunctional acidic catalyst is in a deep eutectic state. The method includes adding hemicellulose or xylose and the bifunctional acidic catalyst as an aqueous phase into a reactor, followed by adding an oil phase extractant, generating furfural in the reactor through catalytic dehydration and hydrolysis reaction, and continuously extracting the furfural from the aqueous phase by the oil phase extractant during the reaction process until the reaction is ended. The method can reduce the activation energy required for the reaction and increase the yield of furfural. Both the aqueous phase and the catalyst may be recycled in the preparation process, which can reduce energy consumption and production cost.

### CROSS-REFERENCE TO RELATED APPLICATIONS

This application is a Continuation of International Application No. PCT/CN2024/095618, filed on May 27, 2024, which claims priority to Chinese Patent Application No. 202310907836.9, filed on Jul. 23, 2023, the entire contents of each of which are hereby incorporated by reference.

### TECHNICAL FIELD

The present disclosure relates to the technical field of furfural preparation, and in particular, to bifunctional acidic catalysts and preparation methods of furfural via catalyzing hemicellulose or xylose.

### BACKGROUND

Lignocellulosic biomass is a renewable carbon resource abundant in agricultural wastes, municipal solid wastes, and algae. Lignocellulosic biomass is mainly composed of cellulose, hemicellulose, and lignin. Hemicellulose is primarily obtained by highly branching and polymerization of pentose sugars (e.g., xylans and arabinoxylans). In the presence of an acidic catalyst, hemicellulose is first depolymerized to monosaccharides (e.g., xylose and arabinose) and then further dehydrated to generate furfural.

Furfural is an important renewable platform chemical widely used in the production of various furan derivatives. Furfural can also undergo condensation reaction with aldehydes and ketones to form oxygenated intermediates, which are subsequently subjected to hydrodeoxygenation to produce liquid alkanes used directly as liquid fuels. While furfural has significant potential in the chemical industry, current production processes face challenges such as a low yield and environmental pollution. To further reduce the cost of furfural production, it is essential to develop a sustainable production process.

Currently, the catalysts used for furfural preparation by dehydration of hemicellulose or xylose include zeolites, solid acid catalysts, and ionic liquid catalysts. On the one hand, zeolite and solid acid catalysts involve a complicated preparation process with a high activation energy and corrosion of chemical equipment, which is not conducive to long term production. On the other hand, the ionic liquid catalysts operate in the homogeneous reaction system during the catalytic process, making it difficult to separate the catalyst from the reaction system after ending the reaction, resulting in resource waste.

In addition, the existing process for preparing furfural from biomass raw material uses a deep eutectic solvent composed of choline chloride and Lewis acid as a catalyst. Although the process achieves relatively a high yield of furfural, the reaction temperature is very high, basically exceeding 180° C. Other process uses a deep eutectic solvent composed of choline chloride and Brønsted acid as a catalyst, the reaction temperature is low, generally not exceeding 150° C., but the yield of furfural is also low. Another process uses a mixture of Lewis acid and Brønsted acid as a catalyst, the process achieves a high yield of furfural, and the reaction temperature is moderate. However, the catalyst is difficult to separate, resulting in resource waste.

Therefore, there is a need to develop a low-cost, highly catalytically active, and sustainable catalyst for the preparation of furfural by the dehydration of hemicellulose or xylose.

### SUMMARY

The technical problem to be solved by the present disclosure aims to provide a bifunctional acidic catalyst and a preparation method of furfural via catalyzing hemicellulose or xylose. The present disclosure employs a choline chloride combined with Lewis acid and Brønsted acid to form a deep eutectic solvent as a bifunctional acidic catalyst, which is used in the process of preparation furfural via catalyzing hemicellulose or xylose, reducing the activation energy required for the reaction and increasing the yield of furfural. Additionally, both the aqueous phase and the catalyst in the preparation process may be recycled, which reduces energy consumption and production cost.

One or more embodiments of the present disclosure provide a bifunctional acidic catalyst including choline chloride, Brønsted acid, and Lewis acid in a molar ratio within of 1:1:(1-4), the bifunctional acidic catalyst being in a deep eutectic state.

In some embodiments, the Brønsted acid may be any one of oxalic acid, malonic acid, succinic acid, and citric acid.

In some embodiments, the Brønsted acid may be oxalic acid.

In some embodiments, the Lewis acid may be any one of CrCl₃·6H₂O, AlCl₃·6H₂O, SnCl₄·5H₂O, and FeCl₃·6H₂O.

In some embodiments, the Lewis acid may be CrCl₃·6H₂O.

In some embodiments, the bifunctional acidic catalyst may be a deep eutectic solvent in the eutectic state and the deep eutectic solvent is prepared by heating a mixture of choline chloride, oxalic acid, and CrCl₃·6H₂O in a molar ratio of 1:1:1.

In some embodiments, a temperature of the heating may be 70° C. and a time of the heating is 6 h.

One or more embodiments of the present disclosure provide a preparation method of furfural via catalyzing hemicellulose or xylose. The preparation method may include adding hemicellulose or xylose and the bifunctional acidic catalyst as an aqueous phase to a reactor, followed by adding an oil phase extractant, generating furfural in the reactor through catalytic dehydration and hydrolysis reaction, and continuously extracting the furfural from the aqueous phase during the reaction process by the oil phase extractant until the reaction is ended.

In some embodiments, the oil phase extractant may include any one of methyl isobutyl ketone, toluene, tetrahydrofuran, valerolactone, n-butanol, acetophenone, ethyl acetate, and tetrahydronaphthalene.

In some embodiments, a mass concentration of the hemicellulose or xylose may be within a range of 0.05 g/mL-0.25 g/mL in the aqueous phase.

In some embodiments, a mass concentration of the bifunctional acidic catalyst may be within a range of 0.05 g/mL-0.25 g/mL in the aqueous phase.

In some embodiments, an amount of the aqueous phase may be ½ to 1/10 of an amount of the oil phase extractant.

In some embodiments, a reaction temperature of the catalytic dehydration and the hydrolysis reaction may be within a range of 140° C.-160° C.

In some embodiments, a reaction time of the catalytic dehydration and the hydrolysis reaction may be within a range of 30 min-150 min.

In some embodiments, the bifunctional acidic catalyst includes choline chloride, Brønsted acid, and Lewis acid in a molar ratio within a range of 1:1:(1-4) in a deep eutectic state.

The preparation method of furfural via catalyzing hemicellulose or xylose includes adding hemicellulose or xylose and the bifunctional acidic catalyst as an aqueous phase to a reactor, followed by adding an oil phase extractant, generating furfural in the reactor through catalytic dehydration and hydrolysis reaction, and continuously extracting the furfural from the aqueous phase by the oil phase extractant during the reaction process until the reaction is ended. The present disclosure employs choline chloride combined with Lewis acid and Brønsted acid to form a deep eutectic solvent as the bifunctional acidic catalyst, which is used in the process of preparation of furfural via catalyzing hemicellulose or xylose, reducing the activation energy required for the reaction, increasing the yield of furfural, and having a mild reaction condition. Additionally, the aqueous phase and the catalyst may be recycled in the preparation process, which reduces energy consumption and production cost.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a chart illustrating effects of a reaction temperature and a reaction time on a yield of furfural according to example 3 and example 4 of the present disclosure;

FIG. 2 is a chart illustrating effects of a mass concentration of a catalyst B on a yield of furfural according to example 5 of the present disclosure;

FIG. 3 is a chart illustrating effects of a mass concentration of xylose on a yield of furfural according to example 6 of the present disclosure;

FIG. 4 is a chart illustrating effects of a reaction temperature and a reaction time on a yield of furfural according to example 9 and example 10 of the present disclosure; and

FIG. 5 is a chart illustrating effects of a mass concentration of a catalyst B on a yield of furfural according to example 12 in the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

As shown in the present disclosure and claims, the words “one”, “a”, “a kind” and/or “the” are not especially singular but may include the plural unless the context expressly suggests otherwise. In general, the terms “comprise”, “comprises”, “comprising”, “include”, “includes”, and/or “including” merely prompt to include operations and elements that have been clearly identified, and these operations and elements do not constitute an exclusive listing. The methods or devices may also include other operations or elements.

A bifunctional acidic catalyst refers to a catalyst contains two acids, which is used in the synthesis of furfural.

In some embodiments, the bifunctional acidic catalyst includes choline chloride, Brønsted acid, and Lewis acid in a molar ratio of 1:1:(1-4), the bifunctional acidic catalyst being in a deep eutectic state.

The deep eutectic state refers to a state in which the melting point of the bifunctional acidic catalyst is significantly lower than the melting points of the pure substances such as choline chloride, Brønsted acid, and Lewis acid contained in the bifunctional acidic catalyst.

In some embodiments, the molar ratio of choline chloride, Brønsted acid, and Lewis acid in the bifunctional acidic catalyst may be determined based on actual application scenarios and needs.

In some embodiments, the molar ratio of choline chloride, Brønsted acid, and Lewis acid is 1:1:(1-4).

In some embodiments, the molar ratio of choline chloride, Brønsted acid, and Lewis acid may also be one of 1:1:1, 1:1:1.5, 1:1:2.0, 1:1:2.5, 1:1:3.0, 1:1:3.5, and 1:1:4.

In some embodiments, the molar ratio of choline chloride, Brønsted acid, and Lewis acid may also be one of 1:1:(1-1.5), 1:1:(1-2), 1:1:(1-2.5), 1:1:(1-3), 1:1:(1.5-2), 1:1:(1.5-2.5), 1:1:(1.5-3), 1:1:(2-2.5), 1:1:(2-3), 1:1:(2.5-3), 1:1:(3-3.5), and 1:1:(3.5-4).

In some embodiments of the present disclosure, the catalytic performance of the bifunctional acidic catalyst can be effectively ensured and the melting point of the bifunctional acidic catalyst can be effectively reduced by setting the molar ratio of choline chloride, Brønsted acid, and Lewis acid.

In some embodiments, Brønsted acid included in the bifunctional acidic catalyst is any one of oxalic acid, malonic acid, succinic acid, and citric acid.

In some embodiments, Brønsted acid included in the bifunctional acidic catalyst is oxalic acid.

In some embodiments, Lewis acid included in the bifunctional acidic catalyst is any one of CrCl₃·6H₂O, AlCl₃·6H₂O, SnCl₄·5H₂O, and FeCl₃·6H₂O.

In some embodiments, Lewis acid included in the bifunctional acidic catalyst is CrCl₃·6H₂O.

In some embodiments of the present disclosure, by specifying the specific types of Brønsted acid and Lewis acid, it can ensure that the raw material of the bifunctional acidic catalyst is easily accessible, low cost, and environmentally friendly, which makes the bifunctional acidic catalyst highly suitable for large-scale industrial production and application.

In some embodiments, the bifunctional acidic catalyst is a deep eutectic solvent in a low eutectic state and the deep eutectic solvent is prepared by heating a mixture of choline chloride, oxalic acid, and CrCl₃·6H₂O in a molar ratio of 1:1:1.

In some embodiments, a temperature of the heating is 70° C., and a time of the heating is 6 h.

In some embodiments, the temperature of the heating may also be within a range of 68° C.-72° C.

In some embodiments, the temperature of the heating may also be one of 67° C., 68° C., 69° C., 70° C., 71° C., and 72° C.

In some embodiments, the heating temperature may also be one of ranges of 67° C.-69° C., 67° C.-70° C., 67° C.-71° C., 67° C.-72° C., 68° C.-70° C., 68° C.-71° C., 68° C.-72° C., 69° C.-71° C., 69° C.-72° C., and 70° C.-72° C.

In some embodiments, the time of the heating may also be within a range of 5 h-7 h.

In some embodiments, the time of the heating may also be one of 5 h, 5.5 h, 6 h, 6.5 h, and 7 h.

In some embodiments, the time of the heating may also be one of ranges of 5 h-5.5 h, 5 h-6 h, 5 h-6.5 h, 5 h-7 h, 5.5 h-6 h, 5.5 h-6.5 h, 5.5 h-7 h, 6 h-6.5 h, 6 h-7 h, and 6.5 h-7 h.

In some embodiments of the present disclosure, by setting the ratio of the composition of the bifunctional acidic catalyst, it can ensure the catalytic performance of the bifunctional acidic catalyst, which helps to improve the efficiency of the furfural preparation and yield of the furfural. In addition, it can also ensure that the bifunctional acidic catalyst has good water solubility and is easy to be separated from the oil phase extractant at the end of the reaction, and the bifunctional acidic catalyst is recycled multiple times.

In some embodiments, the preparation method of furfural via catalyzing hemicellulose or xylose includes adding raw material of hemicellulose or xylose and the bifunctional acidic catalyst as an aqueous phase to a reactor, followed by adding an oil phase extractant, generating furfural in the reactor through catalytic dehydration and hydrolysis reaction, and continuously extracting the furfural from the aqueous phase during the reaction process by the oil phase extractant until the reaction is ended.

Hemicellulose or xylose refers to a raw material used to prepare furfural. In some embodiments, hemicellulose or xylose may be a finished raw material or may be obtained by extraction from plant material.

In some embodiments, furfural may be prepared using a two-phase catalytic reaction.

The furfural preparation system includes an aqueous phase and an oil phase extractant. The aqueous phase is used for a continuous reaction to generate furfural, and the oil phase extractant is used to continuously extract furfural from the aqueous phase.

In some embodiments, the raw material of hemicellulose or xylose may be prepared into an aqueous solution and added to the reactor, followed by the addition of the bifunctional acidic catalyst and thorough stirring to obtain an aqueous phase system.

In some embodiments, a mass concentration of hemicellulose or xylose may be within a range of 0.05 g/mL-0.25 g/mL in the aqueous phase.

In some embodiments, the mass concentration of hemicellulose or xylose may also be one of 0.05 g/mL, 0.10 g/mL, 0.15 g/mL, 0.20 g/mL, and 0.25 g/mL in the aqueous phase.

In some embodiments, the mass concentration of hemicellulose or xylose may also be one of the ranges of 0.05 g/mL-0.10 g/mL, 0.05 g/mL-0.15 g/mL, 0.05 g/mL-0.20 g/mL, 0.10 g/mL-0.15 g/mL, 0.10 g/mL-0.20 g/mL, 0.10 g/mL-0.25 g/mL, 0.15 g/mL-0.20 g/mL, 0.15 g/mL-0.25 g/mL, and 0.20 g/mL-0.25 g/mL in the aqueous phase.

In some embodiments, the mass concentration of the bifunctional acidic catalyst may be within a range of 0.05 g/mL-0.25 g/mL in the aqueous phase.

In some embodiments, the mass concentration of the bifunctional acidic catalyst may also be one of 0.05 g/mL, 0.10 g/mL, 0.15 g/mL, 0.20 g/mL, and 0.25 g/mL in the aqueous phase.

In some embodiments, the mass concentration of the bifunctional acidic catalyst may also be one of the ranges of 0.05 g/mL-0.10 g/mL, 0.05 g/mL-0.15 g/mL, 0.05 g/mL-0.20 g/mL, 0.10 g/mL-0.15 g/mL, 0.10 g/mL-0.20 g/mL, 0.10 g/mL-0.25 g/mL, 0.15 g/mL-0.20 g/mL, 0.15 g/mL-0.25 g/mL, and 0.20 g/mL-0.25 g/mL in the aqueous phase.

In some embodiments of the present disclosure, by setting the mass concentration of hemicellulose or xylose and the mass concentration of the bifunctional acidic catalyst, it not only avoids a slow synthesis rate due to an excessively low mass concentration, but also avoids a decrease in yield due to increased side reactions caused by an excessively high mass concentration, thereby ensuring the synthesis rate and the yield.

The oil phase extractant is an organic phase used to extract furfural from the aqueous organic phase.

In some embodiments, the oil phase extractant includes any one of methyl isobutyl ketone, toluene, tetrahydrofuran, valerolactone, n-butanol, acetophenone, ethyl acetate, and tetrahydronaphthalene.

In some embodiments of the present disclosure, by setting the type of the oil phase extractant, it can ensure a good furfural extraction efficiency while the oil phase extractant is sufficiently separated from the aqueous phase.

In some embodiments, an amount of the aqueous phase is ½ to 1/10 of an amount of the oil phase extractant.

In some embodiments of the present disclosure, by setting the ratio of the aqueous phase to the oil phase extractant, it can reduce the retention time of furfural in the aqueous phase and promote the rapid transfer of furfural to the oil phase extractant, ensuring the mass transfer efficiency of furfural from the aqueous phase to the oil phase extractant.

In some embodiments, after adding the aqueous and the oil phase extractant to the reactor, the reaction may be carried out under magnetically stirred and heated conditions. The magnetic stirring speed, the reaction temperature, and the reaction time may be determined based on actual application scenarios and needs.

In some embodiments, the reaction temperature of the catalytic dehydration and hydrolysis reaction may be within a range of 140° C.-160° C., and the reaction time of the catalytic dehydration and hydrolysis reaction may be within a range of 30 min-150 min.

In some embodiments, the reaction temperature of the catalyzed dehydration and hydrolysis reaction may also be one of 140° C., 145° C., 150° C., 155° C., and 160° C.

In some embodiments, the reaction temperature of the catalyzed dehydration and hydrolysis reaction may also be one of the ranges of 140° C.-145° C., 140° C.-150° C., 140° C.-155° C., 145° C.-150° C., 145° C.-155° C., 145° C.-160° C., 150° C.-155° C., 150° C.-160° C., and 155° C.-160° C.

In some embodiments, the reaction time of the catalyzed dehydration and hydrolysis reaction may also be one of 30 min, 45 min, 60 min, 75 min, 90 min, 105 min, 120 min, 135 min, and 150 min.

In some embodiments, the reaction time of the catalyzed dehydration and hydrolysis reaction may also be one of the ranges of 30 min-75 min, 30 min-90 min, 30 min-105 min, 30 min-120 min, 30 min-135 min, 30 min-150 min, 45 min-90 min, 45 min-105 min, 45 min-120 min, 45 min-135 min, 45 min-150 min, 60 min-105 min, 60 min-120 min, 60 min-135 min, 60 min-150 min, 75 min-120 min, 75 min-135 min, 75 min-150 min, 90 min-135 min, 90 min-150 min, and 105 min-150 min.

In some embodiments, the hemicellulose or xylose undergoes catalytic dehydration and hydrolysis reaction to generate furfural in the reactor, and the oil phase extractant continually extracts the furfural from the aqueous phase during the reaction process until the end of the reaction to obtain the furfural-rich oil phase extractant and the aqueous phase.

In some embodiments, after the reaction, the aqueous phase and the furfural-rich oil phase extractant may be separated by liquid separation, and the pure furfural may be obtained by vacuum distillation, evaporation of the extractant, recovery, etc. The aqueous phase at the reaction may be filtered to remove deposits and impurities and be used for the next furfural preparation together with the recovered oil phase extractant.

In some embodiments of the present disclosure, by adopting a two-phase preparation system including an aqueous phase and an oil phase extractant, it can promote the reaction equilibrium in the aqueous phase to move toward the direction of furfural generation by continuously generating the furfural in the aqueous phase and continuously extracting the furfural from the aqueous phase with the oil phase extractant, thus improving the yield of furfural and inhibiting the occurrence of side reaction of furfural in the aqueous phase. After the reaction, due the insolubility between the oil phase extractant and the aqueous phase, the aqueous phase containing the bifunctional acidic catalyst and the furfural-rich oil phase extractant may be separated through a simple liquid separation, which is convenient for the subsequent recycling of the bifunctional acidic catalyst and the oil phase extractant, helps to reduce the cost of the production of furfural, and minimizes the resource waste.

The bifunctional acidic catalysts and the preparation methods of furfural via catalyzing hemicellulose or xylose are further illustrated hereinafter by specific examples.

### Example 1

Preparation of different ratios of bifunctional acidic catalysts included operations S11 to S13 as follows.

In S11, 1.39 g of choline chloride (ChCl), 0.9 g of oxalic acid (Ea), and 2.66 g of CrCl₃·6H₂O, with a molar ratio of 1:1:1, were weighed and placed into a screw-neck bottle, respectively, and heated at 70° C. for 6 h to obtain a bifunctional acidic ionic deep eutectic solvent, which was denoted as a catalyst B.

In S12, by changing the molar ratio in S11 to 1:1:0.5, 1:1:2, 1:1:3, and 1:1:4, bifunctional acidic ionic deep eutectic solvents were obtained according to the steps in S11, which were denoted as catalysts A, C, D, and E.

In S13, by replacing oxalic acid in S11 with malonic acid, succinic acid, and citric acid, respectively, with a molar ratio of 1:1:1, bifunctional acidic catalysts were synthesized according to the steps in S11, which were denoted as catalysts F, G, and H.

In S14, by mixing choline chloride (ChCl) with oxalic acid (Ea) and CrCl₃·6H₂O in a molar ratio of 1:1, respectively, acidic eutectic catalysts were synthesized according to the steps in S11, which were denoted as catalysts M and N.

### Example 2

The first preparation method of furfural via catalyzing hemicellulose or xylose included operations S21 to S23 as follows.

In S21, catalysts A, B, C, D, E, F, G, H, I, J, K, M, and N prepared in example 1 were added to a reactor of 25 mL, an addition amount of the catalysts was 20% of the mass of the aqueous phase, a volume ratio of the oil phase to the aqueous phase was 8:1, and a mass concentration of the added xylose was 0.15 g/mL (the mass concentration being on the basis of the mass of the water, the same hereinafter).

In S22, the reactor was placed in an oil bath pot, heated to 140° C. under magnetic stirring, and reacted for 90 min.

In S23, after the reactor was cooled to room temperature, an upper oil phase and a lower aqueous phase were taken to detect the content of furfural and calculate the yield of furfural, respectively.

The yield of furfural (YFF) was determined when the amount of the added catalyst was 20% of the mass of the aqueous phase, as shown in Table 1 below.

Table 1 Effect of catalysts with different ratios pf raw material on YFF (using xylose as substrate)

TABLE 1
Effect of catalysts with different ratios pf raw material on YFF (using xylose as substrate)

| Number | A | B | C | D | E | F | G | H | I | J | K | M | N |
|---|---|---|---|---|---|---|---|---|---|---|---|---|---|
| YFF (%) | 64.1 | 87.0 | 72.6 | 68.7 | 70.3 | 39.0 | 42.7 | 43.3 | 51.8 | 44.0 | 46.9 | 54.7 | 51.8 |

As can be seen from Table 1, the YFF is the highest at 87.0% when using the catalyst B.

### Example 3

The second preparation method of furfural via catalyzing hemicellulose or xylose included operations S31 to S33 as follows.

In S31, the catalyst B prepared in example 1 was taken and added to a reactor, an addition amount of the catalyst B was 20% of the mass of the aqueous phase, a volume ratio of methyl isobutyl ketone (MIBK) to water was 8:1, and the mass concentration of the added xylose was 0.15 g/mL.

In S32, the reactor was placed in an oil bath pot, heated to 140° C.-160° C. under magnetic stirring, and reacted for 90 min.

In S33, after the reactor was cooled to room temperature, an upper oil phase and a lower aqueous phase were taken to detect the content of furfural and calculate the YFF, respectively using the same manner in example 2.

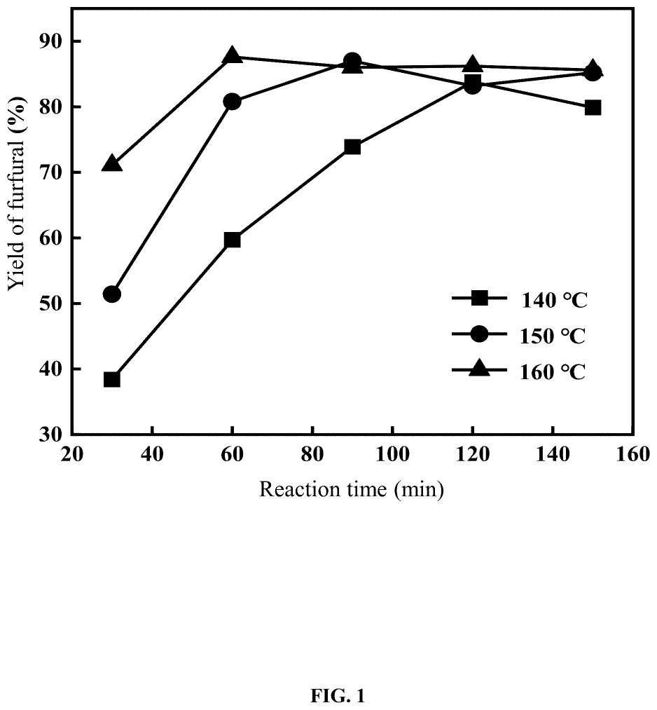

The detection results were shown in FIG. 1. It can be seen from FIG. 1 that when the reaction temperature is 140° C., 150° C., and 160° C., the YFF is 73.9%, 87.0%, and 86.0%, respectively; and when the reaction temperature is 150° C., the YFF is the highest at 87.0%.

### Example 4

The third preparation method of furfural via catalyzing hemicellulose or xylose included operations S41-S42 as follows.

In S41, the catalyst B prepared in example 1 was taken and added to the reactor, an addition amount of the catalyst B was 20% of the mass of the aqueous phase, a volume ratio of MIBK to water was 8:1, and the mass concentration of the added xylose was 0.15 g/mL.

In S42, the reactor was placed in an oil bath pot, heated to 150° C. under magnetic stirring, and reacted for 30 min, 60 min, 90 min, 120 min, and 150 min, respectively; and after the reactor was cooled to room temperature, the upper oil phase and the lower aqueous phase were taken to detect the content of furfural and the YFF, respectively, using the same manner in example 2.

The detection results were shown in FIG. 1. It can be seen from FIG. 1 that the YFF were 51.4%, 80.8%, 87.0%, 83.2%, and 85.2% when the reaction time is 30 min, 60 min, 90 min, 120 min, and 150 min, respectively; and when the reaction time is 90 min, the YFF is the highest at 87.0%.

### Example 5

The fourth preparation method of furfural via catalyzing hemicellulose or xylose included operations S51-S53 as follows.

In S51, the catalyst B prepared in example 1 was weighed according to a mass concentration of 0.05 g/mL, 0.10 g/mL, 0.15 g/mL, 0.20 g/mL, and 0.25 g/mL, respectively, and added to a reactor of 25 mL, a volume ratio of MIBK to water was 8:1, and a mass concentration of the added xylose was 0.15 g/mL.

In S52, the reactor was placed in an oil bath pot, heated to 150° C. under magnetic stirring, and reacted for 90 min.

In S53, after the reactor was cooled to room temperature, an upper oil phase and a lower aqueous phase were taken to detect the content of furfural and calculate the YFF, respectively using the same manner in example 2.

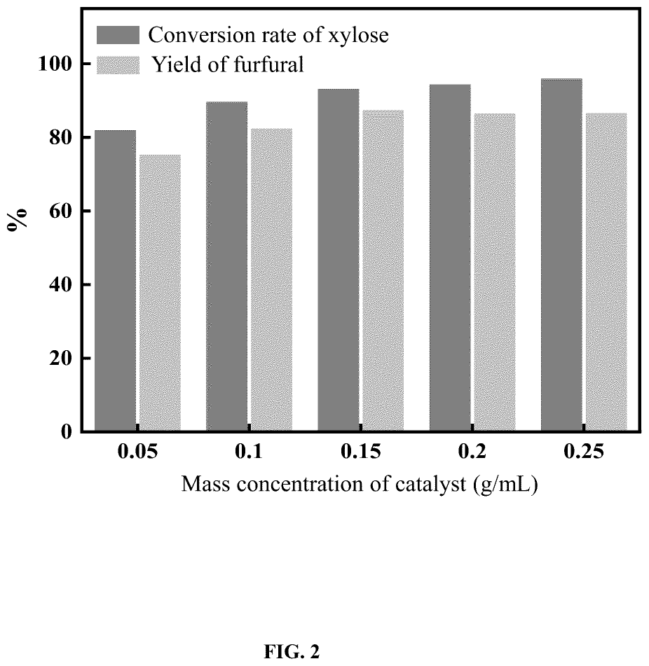

The detection results were as shown in FIG. 2. It can be seen form FIG. 2 that when the mass concentration of the catalyst B is 0.05 g/mL, 0.10 g/mL, 0.15 g/mL, 0.20 g/mL, and 0.25 g/mL, respectively, the YFF is 75.8%, 82.9%, 83.0%, 87.0% and 87.1%, respectively. It is comprehensively considered that the optimal mass concentration of catalyst B is 0.20 g/mL, and the corresponding YFF is 87.0%.

### Example 6

The fifth preparation method of furfural via catalyzing hemicellulose or xylose included operations S61-S63 as follows.

In S61, the catalyst B prepared in example 1 was taken and added to the reactor, a mass concentration of the catalyst B was 0.20 g/mL, and a volume ratio of MIBK to water was 8:1.

In S62, xylose were weighed according to a mass concentration of 0.05 g/mL, 0.10 g/mL, 0.15 g/mL, 0.20 g/mL, and 0.25 g/mL, respectively, and added to the reactor; and the reactor was placed in an oil bath pot, heated to 150° C. under magnetic stirring, and reacted for 90 min.

In S63, after the reactor was cooled to room temperature, an upper oil phase and a lower aqueous phase were taken to detect the content of furfural and calculate the YFF, respectively using the same manner in example 2.

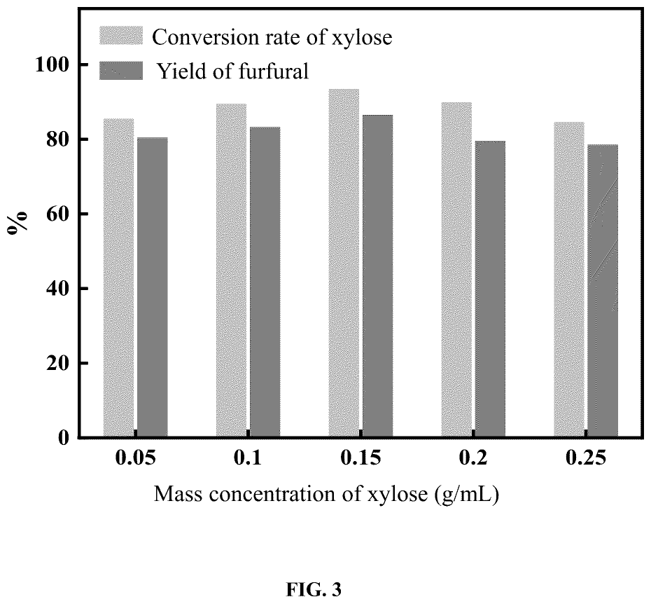

The detection results were shown in FIG. 3. It can be seen from FIG. 3 that when the mass concentration of xylose is 0.05 g/mL, 0.10 g/mL, 0.15 g/mL, 0.20 g/mL, and 0.25 g/mL, respectively, the YFF is 80.9%, 83.7%, 87%, 80%, and 79%, respectively; and when the mass concentration of xylose is 0.15 g/mL, the YFF is the highest at 87.0%.

### Example 7

The sixth preparation method of furfural via catalyzing hemicellulose or xylose included operations S71-S73 as follows.

In S71, the catalyst B prepared in example 1 was taken and added to the reactor, the mass concentration of the catalyst B was 0.20 g/mL, and the mass concentration of the added xylose was 0.15 g/mL.

In S72, MIBK and water were added to the reactor according to a volume ratio of 2:1, 4:1, 6:1, 8:1, and 10:1, respectively, and the reactor was placed in an oil bath pot, heated to 150° C. under magnetic stirring, and reacted for 90 min.

In S73, after the reactor was cooled to room temperature, an upper oil phase and a lower aqueous phase were taken to detect the content of furfural and calculate the YFF, respectively using the same manner in example 2.

It is determined that the YFF is 81.8%, 83.3%, 85.5%, 87%, and 85.9% when the volume ratio of MIBK to water are 2:1, 4:1, 6:1, 8:1, and 10:1, respectively. When the volume ratio of the oil phase to the aqueous phase is 8:1, the YFF is the highest at 87.0%.

### Example 8

The seventh preparation method of furfural via catalyzing hemicellulose or xylose included operations S81-S83 as follows.

In S81: the catalyst B prepared in example 1 was taken and added to the reactor, a mass concentration of the catalyst B was 0.20 g/mL, a mass concentration of the added xylose was 0.15 g/mL, the volume ratio of the oil phase to the aqueous phase was 8:1, and oil phase was MIBK, toluene, tetrahydrofuran, acetophenone, valerolactone, n-butanol, tetrahydronaphthalene, and ethyl acetate, respectively.

In S82, the reactor was placed in an oil bath pot, heated to 150° C. under magnetic stirring, and reacted for 90 min.

In S83, after the reactor was cooled to room temperature, an upper oil phase and a lower aqueous phase were taken to detect the content of furfural and calculate the YFF, respectively using the same manner in example 2.

It is determined that the YFF is 87.0%, 78.1%, 76%, 66%, 53.2%, 49.9%, 69.4%, and 60.2%, respectively, when the oil phase is MIBK, toluene, tetrahydrofuran, acetophenone, valerolactone, n-butanol, tetrahydronaphthalene, and ethyl acetate, respectively. When the oil phase was MIBK, the YFF is the highest at 87.0%.

### Example 9

The eighth preparation method of furfural via catalyzing hemicellulose or xylose included operations S91-S93 as follows.

In S91, the catalyst B prepared in example 1 was taken and added to the reactor, the mass concentration of the catalyst B was 0.20 g/mL, a mass concentration of the added hemicellulose was 0.15 g/mL, and a volume ratio of the oil phase to the aqueous phase was 8:1.

In S92, the reactor was placed in an oil bath pot, heated to 140° C.-160° C. under magnetic stirring, and reacted for 90 min.

In S93, after the reactor was cooled to room temperature, an upper oil phase and a lower aqueous phase were taken to detect the content of furfural and calculate the YFF, respectively using the same manner in example 2.

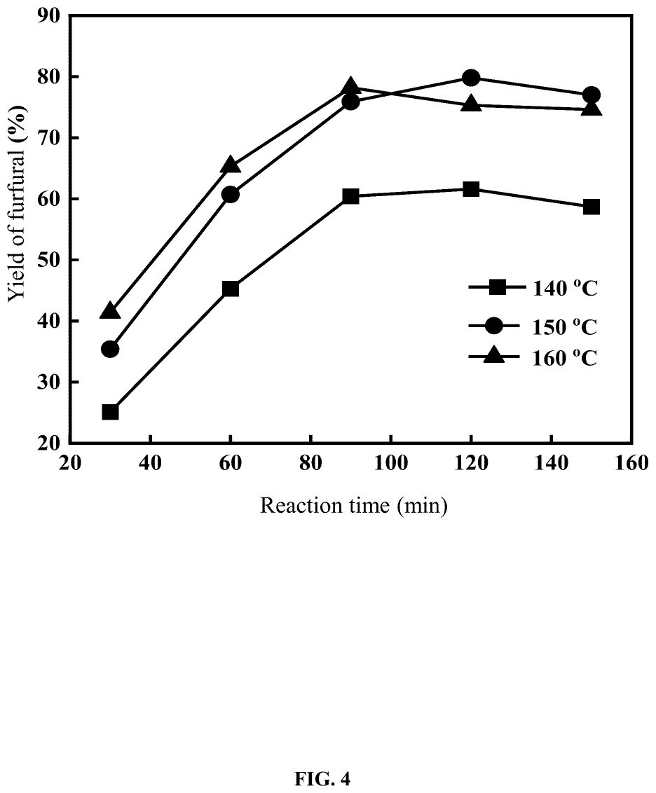

The detection results were as shown in FIG. 4. It can be seen from FIG. 4 that when the reaction temperature is 140° C., 150° C., and 160° C., the YFF is 60.4%, 79.8%, and 78.2%, respectively; and when the reaction temperature is 150° C., the YFF is the highest at 79.8%.

### Example 10

The ninth preparation method of furfural via catalyzing hemicellulose or xylose included operations S101-S103 as follows.

In S101, the catalyst B prepared in example 1 was taken and added to the reactor, the mass concentration of the catalyst B was 0.20 g/mL, and a mass concentration of the added hemicellulose was 0.15 g/mL. A volume ratio of the oil phase to the aqueous phase was 8:1.

In S102, the reactor was placed in an oil bath pot, heated to 150° C. under magnetic stirring, and the reacted for 30 min-150 min.

In S103, an upper oil phase and a lower aqueous phase were taken to detect the content of furfural and calculate the YFF, respectively using the same manner in example 2.

The detection results were determined as shown in FIG. 4. It can be seen from FIG. 4 that when the reaction time is 30 min, 60 min, 90 min, 120 min, and 150 min, respectively, the YFF is 35.4%, 60.7%, 79.8%, 78.2%, and 77%, respectively; and when the reaction time is 90 min, the YFF is the highest at 79.8%.

### Example 11

The tenth preparation method of furfural via catalyzing hemicellulose or xylose included operations S111-S113 as follows.

In S111, hemicellulose was weighed according to a mass concentration of 0.10 kg/L, 0.15 kg/L, 0.2 kg/L, 0.25 kg/L, 0.3 kg/L, 0.35 kg/L, and 0.4 kg/L, respectively, and added to the reactor. The volume ratio of the oil phase to the aqueous phase was 8:1, and the mass concentration of the catalyst B prepared in example 1 was 0.20 g/mL.

In S112, the reactor was placed in an oil bath pot, heated to 150° C. under magnetic stirring, and reacted for 90 min.

In S113, an upper oil phase and a lower aqueous phase were taken to detect the content of furfural and calculate the YFF, respectively using the same manner in example 2.

The yield of furfural prepared by dehydration of hemicellulose with different mass concentrations was determined and shown in Table 2.

TABLE 2
Effect of hemicellulose with different mass concentrations
on YFF (using hemicellulose as substrate)

| | Mass concentration (g/mL) | | | | | | |
|---|---|---|---|---|---|---|---|
| | 0.10 | 0.15 | 0.2 | 0.25 | 0.3 | 0.35 | 0.4 |
| YFF (%) | 75.1 | 79.8 | 74.9 | 73.4 | 68.7 | 63.8 | 63.4 |

As can be seen from Table 2, when furfural is prepared by dehydration of hemicellulose at the mass concentration of 0.15 g/mL, the YFF is the highest at 79.8%.

### Example 12

The eleventh preparation method of furfural via catalyzing hemicellulose or xylose included operations S121-S123 as follows.

In S121, the catalyst B prepared in example 1 was weighed according to a mass concentration of 0.05 g/mL, 0.10 g/mL, 0.15 g/mL, 0.20 g/mL, and 0.25 g/mL, respectively and added to a reactor of 25 mL. A volume ratio of MIBK to water was 8:1, and a mass concentration of the added hemicellulose was 0.15 g/mL.

In S122, the reactor was placed into an oil bath pot, heated to 150° C. under magnetic stirring, and reacted for 90 min.

In S123, an upper oil phase and a lower aqueous phase were taken to detect the content of furfural and calculate the YFF, respectively using the same manner in example 2.

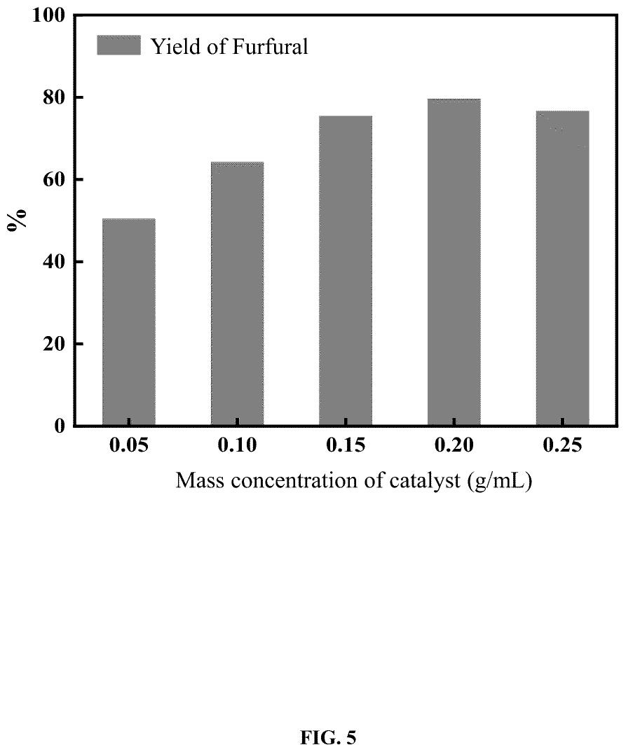

The detection results were shown in FIG. 5. It can be seen from FIG. 5 that when the mass concentration of catalyst B is 0.05 g/mL, 0.10 g/mL, 0.15 g/mL, 0.20 g/mL, and 0.25 g/mL, respectively, the YFF was 50.6%, 64.4%, 75.6%, 79.8%, and 76.8%, respectively. It is comprehensively considered that the optimal mass concentration of catalyst B is 0.20 g/mL, and the corresponding YFF is 79.8%.

### Example 13

The twelfth preparation method of furfural via catalyzing hemicellulose or xylose included operations S131-S133 as follows.

In S131, the catalyst B prepared in example 1 was taken and added to the reactor, a mass concentration of the catalyst B was 0.20 g/mL, a mass concentration of xylose was 0.15 g/mL, and a volume ratio of MIBK to water was 8:1.

In S132, the reactor was placed in an oil bath pot, heated to 150° C. under magnetic stirring, and reacted for 90 min.

In S133, after the rector was cooled to room temperature, an upper oil phase and a lower aqueous phase were taken to detect the content of furfural and calculate the YFF, respectively using the same manner in example 2.

After the reaction is ended, the whole reaction system in the reactor exhibited two-phase separation of aqueous phase and oil phase. The aqueous phase (containing catalyst) and oil phase were separated by a separatory funnel, and the separated aqueous phase was recycled to form a new two-phase reaction system with the fresh oil phase. The catalytic reaction was carried out repeatedly according to the above operations 131-133 to prepare furfural, and the YFF was determined for each cycle.

It is determined that when the number of cycles of the deep eutectic catalyst is 0, 1, 2, 3, 4, 5, 6, 7, and 8, respectively, the YFF is 87.0%, 88.7%, 88.5%, 87.6%, 85.2%, 86.8%, 85.3%, 86.5%, and 85.7%. It may be found that there is almost no significant loss of activity after recycling the deep eutectic catalyst for 8 cycles.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment”, “one embodiment”, or “an alternative embodiment” in various portions of the present disclosure are not necessarily all referring to the same embodiment. In addition, some features, structures, or characteristics of one or more embodiments in the present disclosure may be properly combined.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about”, “approximate”, or “substantially”. For example, “about”, “approximate”, or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A bifunctional acidic catalyst, comprising choline chloride, Brønsted acid, and Lewis acid in a molar ratio of 1:1:(1-4), wherein the bifunctional acidic catalyst is in a deep eutectic state.

2. The bifunctional acidic catalyst of claim 1, wherein the Brønsted acid is any one of oxalic acid, malonic acid, succinic acid, and citric acid.

3. The bifunctional acidic catalyst of claim 1, wherein the Brønsted acid is oxalic acid.

4. The bifunctional acidic catalyst of claim 1, wherein the Lewis acid is any one of CrCl₃·6H₂O, AlCl₃·6H₂O, SnCl₄·5H₂O, and FeCl₃·6H₂O.

5. The bifunctional acidic catalyst of claim 1, wherein the Lewis acid is CrCl₃·6H₂O.

6. The bifunctional acidic catalyst of claim 1, wherein the bifunctional acidic catalyst is a deep eutectic solvent in the deep eutectic state, wherein the deep eutectic solvent is prepared by heating a mixture of choline chloride, oxalic acid, and CrCl₃·6H₂O in a molar ratio of 1:1:1.

7. The bifunctional acidic catalyst of claim 1, wherein a temperature of the heating is 70° C. and a time of the heating is 6 h.

8. A preparation method of furfural via catalyzing hemicellulose or xylose, comprising:
adding the hemicellulose or the xylose and the bifunctional acidic catalyst of claim 1 as an aqueous phase into a reactor, followed by adding an oil phase extractant into the reactor;
generating the furfural in the reactor through catalytic dehydration and hydrolysis reaction; and
continuously extracting the furfural from the aqueous phase by the oil phase extractant during a reaction process until the reaction is ended.

9. The preparation method of claim 8, wherein the oil phase extractant includes any one of methyl isobutyl ketone, toluene, tetrahydrofuran, valerolactone, n-butanol, acetophenone, ethyl acetate, and tetrahydronaphthalene.

10. The preparation method of claim 8, wherein a mass concentration of the hemicellulose or xylose is within a range of 0.05 g/mL-0.25 g/mL in the aqueous phase.

11. The preparation method of claim 8, wherein a mass concentration of the bifunctional acidic catalyst is within a range of 0.05 g/mL-0.25 g/mL in the aqueous phase.

12. The preparation method of claim 8, wherein an amount of the aqueous phase is ½ to 1/10 of an amount of the oil phase extractant.

13. The preparation method of claim 8, wherein a reaction temperature of the catalytic dehydration and the hydrolysis reaction is within a range of 140° C.-160° C.

14. The preparation method of claim 8, wherein a reaction time of the catalytic dehydration and the hydrolysis reaction is within a range of 30 min-150 min.
